(19) United States
(12) Patent Application Publication
(10) Pub. No.: US 2021/0010985 A1
(43) Pub. Date: Jan. 14, 2021

(54) METHOD FOR DETERMINING THE ANTIOXIDANT CAPACITY OF A BIOLOGICAL SAMPLE AND RELATED KIT

(71) Applicant: FONDAZIONE ISTITUTO ITALIANO DI TECNOLOGIA, Genova (IT)

(72) Inventors: Deborah PEDONE, Genova (IT); Mauro MOGLIANETTI, Genova (IT); Pier Paolo POMPA, Genova (IT)

(21) Appl. No.: 16/980,027

(22) PCT Filed: Mar. 12, 2019
(86) PCT No.: PCT/IB2019/051970

§ 371 Date: Sep. 11, 2020

(30) Foreign Application Priority Data
Mar. 13, 2018 (IT) 102018000003475

(51) Int. Cl. G01N 31/22; G01N 21/78; G01N 21/33; G01N 33/487; G01N 33/14

(52) U.S. Cl. CPC G01N 31/22; G01N 21/78; G01N 33/143; G01N 33/487; G01N 21/33

(57) ABSTRACT

A method for determining the antioxidant power of a sample of a biological fluid or a food is provided. The method involves contacting the sample to be tested with an aqueous solution of palladium nanoparticles, an oxidizing agent, and a chromogenic peroxidase substrate, and detecting the colour of the final solution thus obtained. The colour intensity of the solution is proportional to the antioxidant power of the sample. A kit for carrying out the method is also provided.

The present invention relates to a method and a kit for determining the antioxidant capacity of a biological sample.

The oxidative stress and the power of modulating it through antioxidants have attracted much attention in the scientific community, since numerous scientific studies and epidemiological investigations have linked oxidative stress to various diseases such as cancer, certain neurodegenerative diseases such as Alzheimer disease, and male infertility. In this context, saliva and other biological fluids such as blood, sweat and urine have important diagnostic potential, reflecting the physiological state of the individual at the time of sample collection. In particular, saliva is known to contain molecules produced by the salivary glands together with serum components transported into the saliva by passive diffusion through the capillaries, as well as other material released by the cells.

To date, there is a large body of literature linking the antioxidative state of saliva and other biological fluids to the onset of serious pathologies. The oxidative stress values in saliva and blood have also been shown to be strongly correlated.

The scientific and medical community is therefore focusing on various salivary biomarkers that allow monitoring of the body condition, such as for example the Total Antioxidant Capacity (TAC). TAC is of great interest because it comprises all the components capable of modulating oxidation, including enzyme and non-enzyme molecules, thus representing the net antioxidant power. TAC modifications can be bidirectional and denote systemic or specific changes in the redox homeostasis of certain tissues.

At the same time, there is a growing interest in the role that antioxidants in food play in human health, given that the beneficial effect resulting from the consumption of fruit, vegetables, tea, coffee and cocoa has actually been attributed to antioxidants.

To date, the main methods for determining the antioxidant capacity of a sample of a biological fluid or a food, for clinical or food analysis, are based on the power of an antioxidant to reduce the free radicals generated through a synthetic procedure, reduce specific metal ions (for example, copper, gold, iron), block fluorogenic radical molecules or modify chromogenic substrates (for example: ABTS (2,2′-azino-bis(3-ethylbenzothiazoline-6-sulphonic acid), DPPH (1,1-diphenyl-2-picrylhydrazyl), TMB (3,3′,5,5′-tetramethylbenzidine), or block superoxide anions synthetically produced.

Much attention has been focused on the development of new tests capable of measuring the total antioxidant power of a given biological sample, in order to be able to assess the total antioxidant capacity thereof. The excessive variety of the available tests, however, has produced a lack of consistency among the results obtainable with the different systems, with a consequent lack of homogeneity and fragmentation of the results. Often, it is also necessary to resort to the use of multiple tests to assess the overall real antioxidant power of a sample.

There is also a lack of tests that lend themselves to be implemented through portable devices, point-of-care devices, and devices executable by the end user. A test with these characteristics would allow a continuous and effective monitoring of the antioxidant power of the organism through the main biological fluids.

An ideal standard method should possess essentially all of the following characteristics:
measure the sample directly, without purification steps;
be simple and low cost;
have a clear physical-chemical mechanism of operation;
not require specific instrumentation;
be reproducible and reliable;
be stable in ambient conditions;
measure both lipophilic and hydrophilic antioxidants;
be usable for studies on large numbers of samples.

The methods developed so far fall into two main categories: Hydrogen Atom Transfer (HAT) and Single Electron Transfer (SAT). Methods belonging to the first category measure the ability of an antioxidant to block the free radicals through the donation of hydrogen. Instead, SAT methods measure the ability of an antioxidant to transfer an electron to reduce a compound, whether it is a metal, a carbonyl or a radical.

The most common HAT methods are:
Total radical-trapping antioxidant parameter (TRAP)
Oxygen radical absorbance capacity
Inhibition of induced LDL oxidation
Total oxyradical scavenging capacity assay (TOSCA).

The most common SET methods are:
Trolox equivalence antioxidant capacity (TEAC) assay
Ferric ion reducing antioxidant power (FRAP) assay
Cupric ions reducing antioxidant power (CUPRAC)
Total antioxidant potential assay with Cu-complex as oxidant
2,2-Diphenyl-1-picrylhydrazyl radical (DPPH) scavenging
2,2-Azinobis 3-ethylbenzthiazoline-6-sulphonic acid radical (ABTS) scavening assay
N,N-dimethyl-p-phenylenediamine radical (DMPD) scavening assay

In addition to the two categories mentioned above, there are also methods that selectively measure the ability of a given substance to block certain biologically relevant oxidants. The most important of these are: Hydrogen peroxide scavening assay, Superoxide anion radical scavening assay, and Hydroxyl radical scavening assay.

New methods for determining the antioxidant power of fluids have been emerging in recent years and consist in the use of gold nanoparticles as a colorimetric sensor. These methods are based on the reduction of gold ions into nanoparticles with the consequent generation of colour associated with the formation of metal nanoparticles. The property of antioxidants to increase the size of the gold nanoparticles and hence their optical properties has also been proposed as a method of detecting antioxidants.

Another method uses the inhibition of the formation of gold nanoparticles by hydrogen peroxide, a phenomenon that results in a lack of staining of the solution. A method also based on gold nanoparticles and their optical properties has further been proposed for detecting antioxidants, which is based on the clustering of colloidal gold nanoparticles and the consequent colour change of the solution.

A further known method for detecting antioxidants in food uses cerium nanoparticles and is based on the change in colour of the nanomaterial upon contact with the food in the liquid phase.

Some of the known methods for measuring the antioxidant power of a biological fluid or a food are based on redox reactions catalysed by antioxidant enzymes (e.g. peroxidase and catalase). However, the latter have high isolation and purification costs, and are extremely sensitive to proteases, pH and temperature. Therefore, the scientific interest is to develop “artificial enzymes”, designated as nanozymes, including palladium nanoparticles, which are ideal for their efficient and selective catalase and peroxidase activities.

Palladium nanoparticles are obtained by simple and inexpensive synthesis and purification protocols, are stable, maintain the catalytic activity unchanged even under extreme temperature and pH conditions, and resist the action of proteases. They are also able to oxidise chromogenic peroxidase substrates (in particular 3,3′,5,5′-tetramethylbenzidine (TMB), 3,3′-diaminobenzidine (DAB)) in the presence of hydrogen peroxide as the oxidizing agent, with a higher affinity for TMB than biological peroxidase.

In the literature, there are few examples of colorimetric tests based on the use of palladium nanoparticles for the detection of biomarkers.

One of the first biological assay based on the use of palladium nanoparticles was proposed by Jianming Lan, et al., Colorimetric determination of sarcosine in urine samples of prostatic carcinoma by mimic enzyme palladium nanoparticles, Analytica Chimica Acta, Volume 825, 2014, doi.org/10.1016/j.aca.2014.03.040, who developed a sarcosine detection system based on the detection by means of TMB and palladium nanoparticles of the hydrogen perodixe produced by the enzyme sarcosine oxidase upon sarcosine degradation.

Nanoparticles consisting of palladium and iron deposited on three-dimensional structures of graphene were instead proposed as substitutes of peroxidase in an assay for the colorimetric determination of glucose level in urine, based on the use of glucose oxidase, TMB and H₂O₂ (Xuejing Zheng, et al., In Situ Synthesis of Self-Assembled Three-Dimensional Graphene-Magnetic Palladium Nanohybrids with Dual-Enzyme Activity through One-Pot Strategy and Its Application in Glucose Probe, ACS Applied Materials & Interfaces 2015 7 (6), 3480-3491, DOI: 10.102/am508540x).

In the clinical field, instead, palladium nanoparticles functionalized with folic acid, associated with the TMB chromogen, have been used for the colorimetric detection of tumour cells, characterized by a higher expression of the folic acid receptor.

However, none of the currently available assays allows the antioxidant power to be assessed through a point-of-care, naked-eye determination, without the need of specific instrumentation.

These and other needs are met by the present invention, which provides a method for determining the antioxidant power of a sample of a biological fluid or a food which, compared to the techniques described in the state of the art, requires very short times (about 5 minutes), is based on the inhibition of colour development by antioxidants present in the sample itself, requires no pretreatment of the sample, and is extremely low cost. Moreover, the detection of the signal, i.e. the colour, can be performed with the naked eye and therefore on the spot (“point-of-care”), without necessarily requiring the use of specific instrumentation.

The method of the invention is as defined in appended claim 1. The scope of the invention also includes a kit as defined in appended claim 11, suitable for implementing the method of the invention.

Further features and advantages are defined in the dependent claims.

The claims form an integral part of the present specification.

The term “palladium nanoparticles” used in the present disclosure means that the nanoparticles do not contain metals other than palladium.

In the method of the present invention, the palladium nanoparticles perform a catalytic function. The reaction substrate is the chromogen TMB (3,3′,5,5′-Tetramethylbenzidine). In the reaction, hydrogen peroxide is typically used as the oxidation agent. The substrate of the oxidation reaction is preferably the chromogen TMB (3,3′,5,5′-tetramethylbenzidine). Hydrogen peroxide is typically used as the oxidizing agent.

The method of the invention is based on the fact that the antioxidant substances in the sample under examination interact with hydrogen peroxide, causing a partial or total inhibition of the TMB oxidation reaction. The result is a less intense development of the blue colour, which, as is known, is formed by oxidation of the TMB chromogenic substrate. It is important to note that the decreased oxidation of TMB by hydrogen peroxide is directly proportional to the amount of antioxidant substances and can therefore be used to colorimetrically quantify the concentration of antioxidants in the sample under examination. This reaction scheme is particularly effective and specific since it allows the total antioxidant capacity of a sample to be measured without the need of any preliminary purification step.

The assay method of the present invention can be applied to several biological fluids such as saliva, blood, sweat, or urine. It can also be applied to liquid food such as fruit juices and edible oils. In the case of oils, it is preferable that the sample is first mixed with a solution of methanol and isopropanol, to which the above listed reagents are subsequently added, without however requiring separation or purification.

The examples that follow are provided for illustration purposes and make reference to the appended drawings, in which:

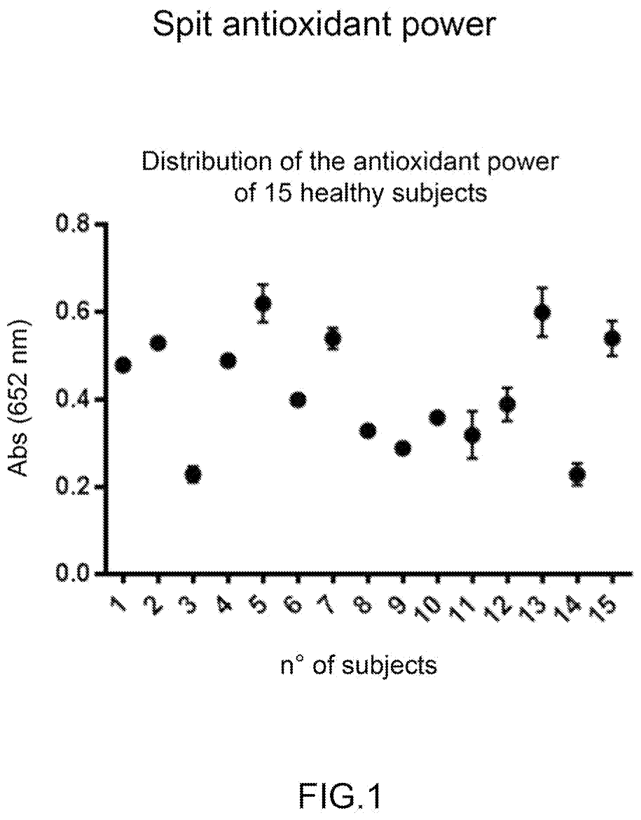

FIG. 1 shows the results of a test performed by UV-visible spectroscopy on 15 samples of saliva obtained from healthy volunteers;

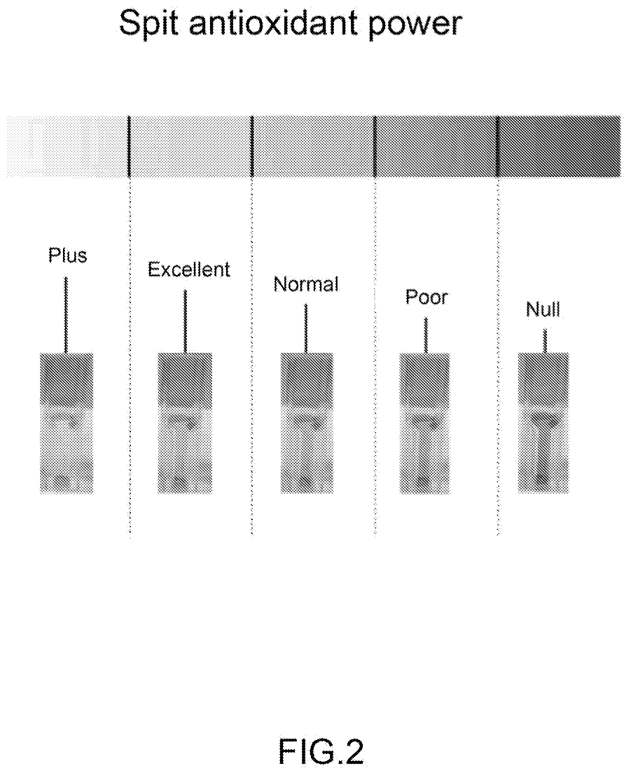

FIG. 2 shows a representative image of the results of the colorimetric method to the naked eye according to the invention performed on 5 samples of saliva (the colour scale at the top is a guide for the interpretation of the test);

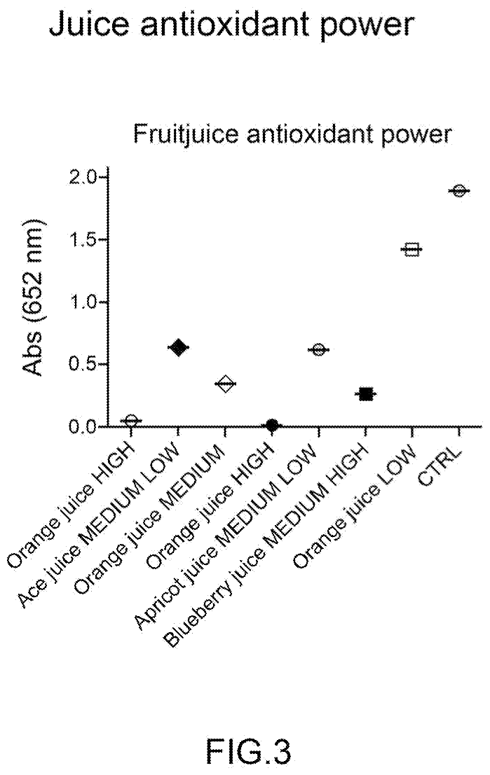

FIG. 3 shows the results of a test carried out by UV-visible spectroscopy on food samples of fruit juice and other industrial beverages;

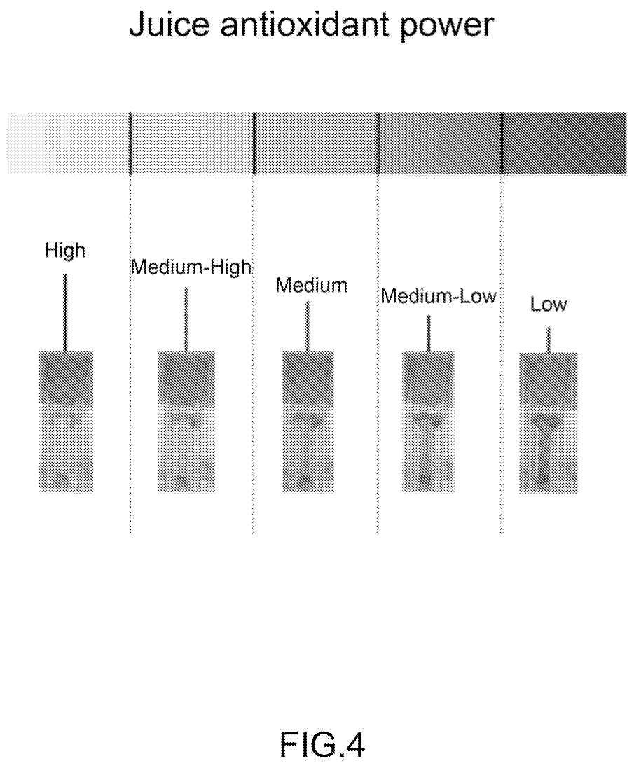

FIG. 4 shows a representative image of the results of the colorimetric method to the naked eye according to the invention performed on 5 samples of fruit juice (the colour scale at the top is a guide for the interpretation of the test).

The examples are provided purely by way of illustration and not of limitation of the scope of the invention as defined by the appended claims.

### EXAMPLES

The following assay is performed:

400 microlitres of an acetate buffer solution, typically between 0.01 and 1 M, preferably between 0.05 and 0.3 M were added to a test tube; the pH can vary between 1 and 7, preferably between 3 and 5.5;

200 microlitres of a TMB (3,3′,5,5′-tetramethylbenzidine) solution were added at a concentration of between 0 and 1 M, preferably between 0.002 and 0.05 M;

100 microlitres of a solution containing palladium nanoparticles were added at a concentration comprised between 0.01 and 1000 ppm palladium, preferably between 0.1 and 10 ppm; the diameter of the nanoparticles may vary between 0.1 nm and 1000 nm, preferably between 1 and 100 nm;

18.3 microlitres of the sample to be tested were added;

the colour development reaction (blue) was initiated by the addition of 533 microlitres of a 1 M hydrogen peroxide solution.

FIG. 1 shows the results obtained by performing the test on 15 saliva samples obtained from healthy volunteers and carrying out the detection by UV-Vis spectroscopy (measurement of the absorbance at 652 nm).

As an alternative to the spectroscopic method, it is possible to carry out the detection by the naked eye, using a reference colour scale which shows different colour intensity bands and the related antioxidant score (e.g., “excellent”, “medium-high”, “standard”, “medium-low”, “low”, cf. FIG. 2).

As indicated above, the method of the invention can also be used to analyse food samples such as fruit juices. FIG. 3 shows the results obtained by testing food samples of fruit juice and other industrial beverages and performing the detection by UV-Vis spectroscopy (measurement of the absorbance at 652 nm).

As an alternative to the spectroscopic method, it is possible to carry out the detection by the naked eye, using a reference colour scale which shows different colour intensity bands and the related antioxidant score (e.g., “excellent”, “medium-high”, “standard”, “medium-low”, “low”, cf. FIG. 4).

What is claimed is:

1. A method for determining the antioxidant power of a sample of a biological fluid or a food, the method comprising:
contacting the sample with an aqueous solution of palladium nanoparticles, an oxidizing agent, and a chromogenic peroxidase substrate; and
detecting colour intensity of a final solution thereby obtained, the colour intensity being proportional to the antioxidant power of the sample.

2. The method of claim 1, wherein the chromogenic peroxidase substrate is 3,3′,5,5′-tetramethylbenzidine (TMB).

3. The method of claim 1, wherein the oxidizing agent is hydrogen peroxide.

4. The method of claim 1, wherein the palladium nanoparticles have a diameter ranging from 0.1 nm to 1000 nm.

5. The method claim 1, wherein the final solution is prepared in a buffer solution having a pH comprised between 1 and 7.

6. The method of claim 1, wherein the colour intensity of the final solution is detected by the naked eye.

7. The method of claim 1, wherein the colour intensity of the final solution is detected by UV-visible spectroscopy.

8. The method of claim 7, wherein the colour intensity of the final solution is detected by measuring absorbance at a wavelength between about 600 and 700 nm.

9. The method of claim 1, wherein the biological fluid is selected from the group consisting of: saliva, blood, sweat, and urine.

10. The method of claim1, wherein the food is a fruit juice.

11. A kit for determining the antioxidant power of a sample of a biological fluid or a food, the kit comprising a chromogenic peroxidase substrate and an aqueous solution of palladium nanoparticles, and optionally further comprising an oxidizing agent and/or a buffer solution, the chromogenic peroxidase substrate being 3,3′,5,5′-tetramethylbenzidine (TMB), the palladium nanoparticles having a diameter ranging from 0.1 nm to 1000 nm, the optional oxidizing agent being hydrogen peroxide and the optional buffer solution being a buffer having a pH comprised between 1 and 7.

12. The kit of claim 11 further comprising:
a predetermined amount of the aqueous solution of palladium nanoparticles at a concentration ranging from 0.01 ppm to 1000 ppm; and
a predetermined amount of a 3,3′,5,5′-tetramethylbenzidine (TMB) solution at a concentration ranging from 0.001 M to 1 M, preferably of from 0.002 M to 0.05 M; and optionally
a predetermined amount of hydrogen peroxide at a concentration comprised between 0.1 M and 10 M; and/or
a predetermined amount of an acetate buffer solution at a concentration comprised between 0.01 M and 1 M, having a pH value comprised between 1 and 7.

13. An in vitro diagnostic method for assessing oxidative stress in a subject, the in vitro diagnostic method comprising:
determining the antioxidant power of a biological fluid sample from the subject by contacting the biological fluid sample with an aqueous solution of palladium nanoparticles, an oxidizing agent, and a chromogenic peroxidase substrate, and
detecting colour intensity of a final solution thereby obtained, the colour intensity being proportional to the antioxidant power of the biological fluid sample, wherein a decreased antioxidant power of the biological fluid sample from the subject compared to a reference sample or value is indicative of the oxidative stress of the subject.

14. The in vitro diagnostic method of claim 13, wherein the subject is suspected of conducting or conducts a health-damaging lifestyle, including alcohol abuse or unhealthy diet.

15. The method of claim 1, wherein the palladium nanoparticles have a diameter ranging from 1 to 100 nm.

16. The method of claim 1, wherein the final solution is prepared in an acetate buffer.

17. The method of claim 1, wherein the food is an oil.

18. The in vitro diagnostic method of claim 13, wherein the subject is suffering or is suspected to be suffering from a disease selected from the group consisting of kidney damage, gout, endometriosis, diabetes and cancer.
